(19) United States
(12) Patent Application Publication
(10) Pub. No.: US 2006/0161259 A1
(43) Pub. Date: Jul. 20, 2006

(54) Knee joint prosthesis

(76) Inventors: Cheng-Kung Cheng, Taipei City (TW); Chang-Hung Huang, Douliou City (TW)

Correspondence Address: BIRCH STEWART KOLASCH & BIRCH, PO BOX 747, FALLS CHURCH, VA, 22040-0747, US

(21) Appl. No.: 11/036,375

(22) Filed: Jan. 18, 2005

(51) Int. Cl. A61F 2/38

(52) U.S. Cl. 623/20.27; 623/20.33

(57) ABSTRACT

An improved knee joint prosthesis located between a thighbone and a tibia to couple with a femur implanted component to allow a patient to resume normal movement and exercise. The knee joint prosthesis adopts a separate design, and includes a tibial insert which has a moving aperture to hold a bracing member coupled with the femur implanted member and a tibial baseplate which has a retaining member. The bracing member and the retaining member are swivelable in the moving aperture, and the bracing member is made from wearing resistant material, so that sliding and wearing that might otherwise occur to the tibial insert may be prevented. Therefore the life span of the posterior stabilized knee joint prosthesis increases.

### FIELD OF THE INVENTION

The present invention relates to an improved knee joint prosthesis and particularly to a prosthetic knee joint to bridge a thighbone and a tibia and to couple with a femur implanted component on the thighbone to enable a patient to resume normal movement and exercise.

### BACKGROUND OF THE INVENTION

The knee joint prosthesis is first developed by Gunston et al in 1968. It mainly aims to substitute an impaired or degenerated knee joint that cannot function normally. It is made of metal to imitate the condyle of the femur and uses ultra high molecular weight polyethylene (UHMWPE) to substitute the function of knee joint and meniscus cartilage. In 1970s, varying prosthetic knee joints have been developed. Development of the knee prosthesis generally follows two directions. One recommends scarifying the posterior cruciate ligament (PCL) during surgical operation. The other one suggests retaining the posterior cruciate ligament during the surgical operation. Above mentioned knee prostheses are designed with different characteristics. Posterior Cruciate Ligament retained knee prosthesis (CR knee prosthesis) could have good clinical results in long term survival analyses but surgeons should pay more attention to make sure whether the PCL has well function during operation.

In terms of the posterior cruciate ligament substituted knee prosthesis (PS knee prosthesis), PCL is scarified to facilitate surgical approaches. Ligament's function is substituted for an artificial mechanism of a tibial post and a femoral cam. It could provide anteriopostrior stability of the knee joint and recover normal knee kinematics after knee joint replacement. Based on above advantages, PS knee prostheses became more reliable and acceptable since 1990. One of the modern techniques is U.S. Pat. No. 4,213,209. It discloses a knee joint prosthesis which includes a tibial baseplate made of metal and implanted in a tibia, a tibial insert made from polyethylene to be coupled on the tibial baseplate and a femur implanted component made of metal and implanted in the thighbone for moving on the concavities of the tibial insert. Knee joint stability is mainly supported through a bracing member integrally formed with the tibial insert that is coupled with the femur implanted component. When the patient moves or walks, the thighbone drives the femur implanted member to move on the concavities of the tibial insert about the bracing member so that the thighbone and the tibia can maintain a flexuous condition to enable the knee joint of the patient to move like before operation. However, clinical diagnoses over the years show that such type of prosthetic knee joint still has drawbacks that affect its life span, notably:

1. As the femur implanted component is made of metal, while the tibial insert is made from polyethylene, when the femur implanted component swivels is coupled with the bracing member and swivels like screw-home mechanism, due to the difference of materials and the fixed bracing member of the tibial insert, the bracing member (namely post structure) on the tibial insert wears off rapidly and even fractures.

2. Besides the impact of the coupling mechanism set forth above, movement of the femur implanted component will cause the tibial insert to slide slightly on the tibial. As the tibial component is made of metal, backside tibial insert wearing also occurs to the lower surface of the tibial insert. These wearing phenomena not only shorten the life span of the prosthetic knee joint but also produce polyethylene debris, induce osteolysis, and lead components loosening.

### SUMMARY OF THE INVENTION

The primary object of the invention is to solve the aforesaid disadvantages. The invention employs a separate design and has a moving aperture on a tibial insert to hold a bracing member which is coupled with a femur implanted component. A tibial insert is provided that has a retaining member. The bracing member and the retaining member may be swiveled in the moving aperture. The bracing member is made from a wearing-resistant material and can prevent the tibial insert from sliding and wearing off, thus the life span of the prosthetic knee joint increases.

The foregoing, as well as additional objects, features, and advantages of the invention will be more readily apparent from the following detailed description, which proceeds with reference to the accompanying drawings.

### BRIEF DESCRIPTION OF THE DRAWINGS

FIG. 1 is a perspective view of the invention.

FIG. 2 is an exploded view of the invention.

FIG. 3 is a sectional view of the invention in an assembled condition.

FIG. 4 is a top view of the invention in an assembled condition.

FIG. 5 is a schematic view of the invention in a use condition.

FIG. 6 is an exploded view of another embodiment of the invention.

FIG. 7 is a sectional view of another embodiment of the invention.

### DETAILED DESCRIPTION OF THE PREFERRED EMBODIMENTS

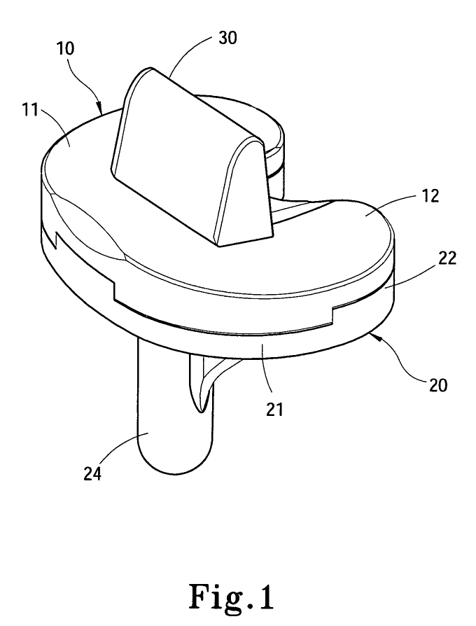

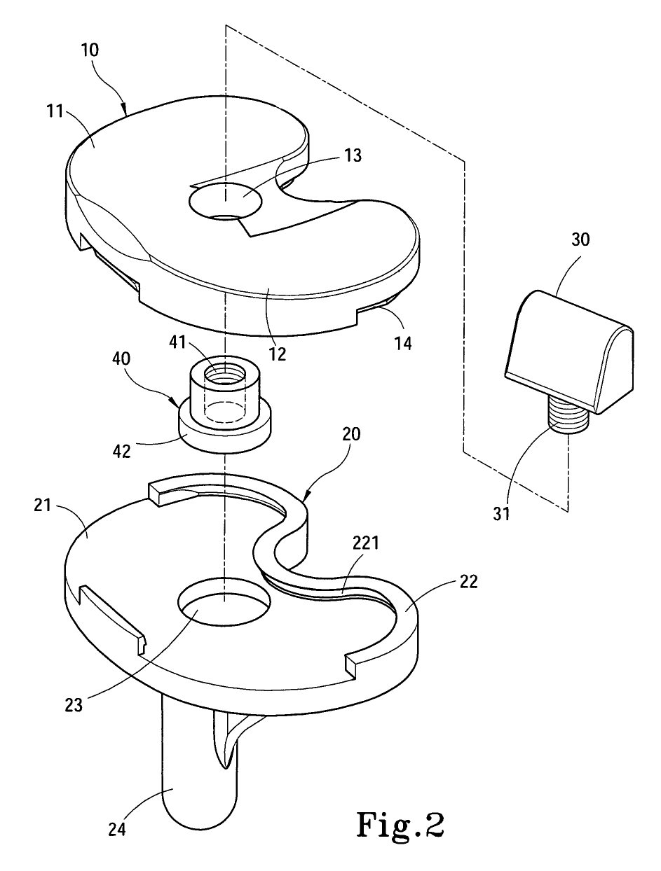

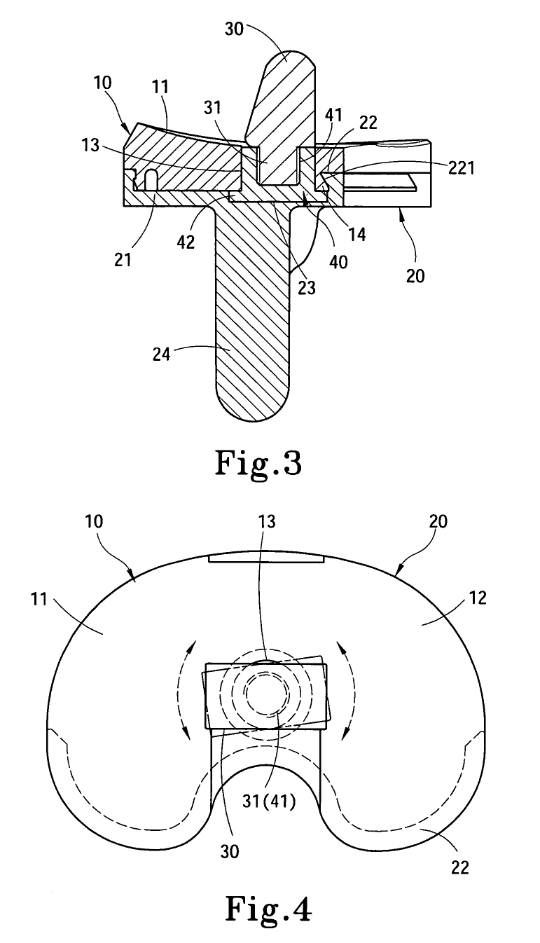

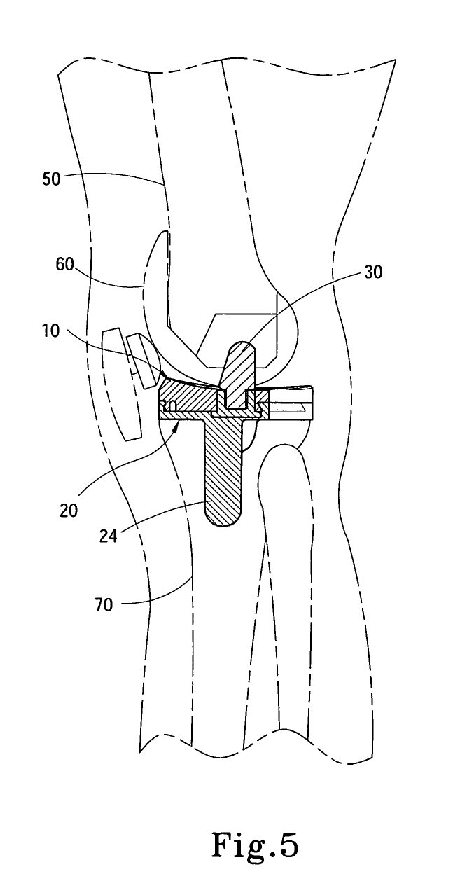

Please refer to FIGS. 1, 2, 3 and 5, the prosthetic knee joint according to the present invention is located between a thighbone 50 and a tibia 70, and is coupled with an femur implanted component 60 fastened to the thighbone 50 to enable a patient to resume normal movements. The prosthetic knee joint includes a tibial insert 10 coupled with the femur implanted component 60 and a tibial baseplate 20 coupled with the tibial insert 10.

The tibial insert 10 has two concavities 11 and 12 that allow the femur implanted component 60 to move thereon. The tibial insert 10 further has a moving aperture 13 formed between the two concavities 11 and 12. The moving aperture 13 holds a bracing member 30 which is coupled with the femur implanted component 60. The bracing member 30 has a fastening section 31.

The tibial baseplate 20 has a holding surface 21 to hold the tibial insert 10, a retaining member 40 which has an anchor section 41 mating the fastening section 31, and an implanting strut 24 extended integrally to couple with the tibia 70.

For assembling the first embodiment set forth above, first, place the tibial insert 10 on the holding surface 21 of the tibial baseplate 20; insert the retaining member 40 in the moving aperture 13. The tibial baseplate 20 has a retaining flange 22 extended from the holding surface 21 on one edge. The retaining flange 22 has an inhibiting section 221 corresponding to an anchor wing 14 formed on the tibial insert 10. Hence the tibial insert 10 is prevented from sliding when the femur implanted component 60 is moved on the two concavities 11 and 12. Moreover, in this embodiment, the bracing member 30 is coupled with the retaining member 40 to swivel in the moving aperture 13. The retaining member 40 has a retaining section 42 which is larger than the moving aperture 13. The tibial dock 20 further has an anchor trough 23 for holding the retaining section 42. The fastening section 31 has external screw threads to engage with internal screw threads formed in the anchor section 41, thus the bracing member 30 and the retaining member 40 may be coupled together and the retaining section 42 is confined by the moving aperture 13 and the anchor trough 23 without escaping. Referring to FIG. 4, when the femur implanted component 60 swivels on the two concavities 11 and 12 about the bracing member 30, due to the separate design of the bracing member 30 and the retaining member 40 and the tibial insert 10, the bracing member 30 and the retaining member 40 may be made from wearing-resistant material without causing serious erosion. Moreover, as the bracing member 30 and the retaining member 40 are moved in the moving aperture 13 and the anchor trough 23, the sliding movement that might otherwise occur to the tibial insert 10 may be prevented from further wearing. Therefore, the life span of the prosthetic knee joint increases.

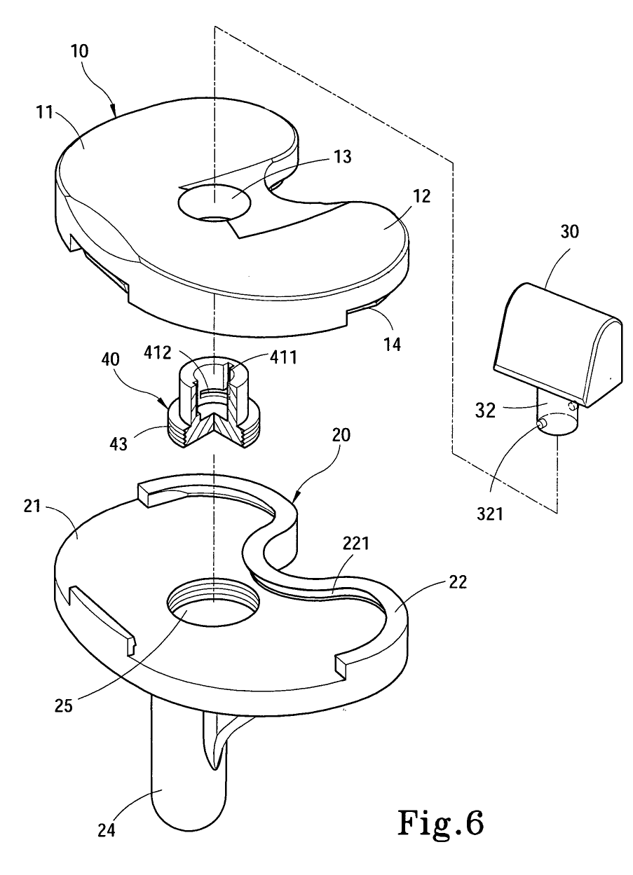

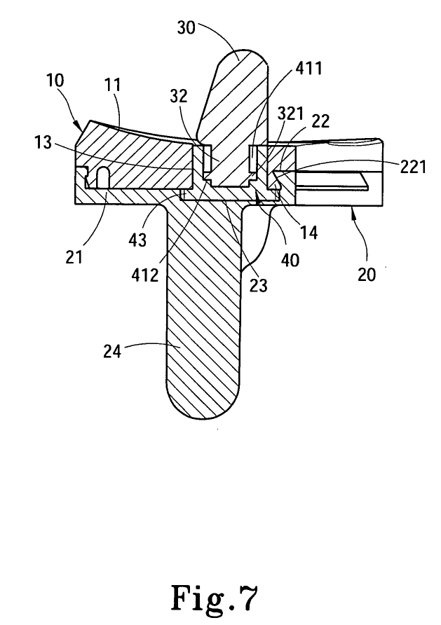

Refer to FIGS. 6 and 7 for another embodiment of the invention. The main feature is that the bracing member 30 and the retaining member 40 are separated so that varying relative movements may be formed between them. This embodiment provides another type of relative moving mechanism. The bracing member 30 has a fastening section 32 which has a moving strut 321. The anchor section 41 on the retaining member 40 has a guiding flute 411 and a moving groove 412 communicating with the guiding flute 411 to receive the moving strut 321. The retaining member 40 and the tibial baseplate 20 may be formed in an integrated manner. Or the retaining member 40 may have an anchor section 43 corresponding to an anchor trough 25 formed on the tibial baseplate 20. The anchor section 43 has external screw threads engageable with internal screw threads formed on the anchor trough 25. When the retaining member 40 is coupled with the tibial baseplate 20, the moving strut 321 slides through the guiding flute 411 into the annular moving groove 412. Therefore when the femur implanted component 60 drives the bracing member 30, it swivels through the fastening section 32 in the moving groove 412. As the bracing member 30 is moved in the retaining member 40, sliding of the tibial insert 10 may be prevented from wearing.

While the preferred embodiments of the invention have been set forth for the purpose of disclosure, modifications of the disclosed embodiments of the invention as well as other embodiments thereof may occur to those skilled in the art. Accordingly, the appended claims are intended to cover all embodiments which do not depart from the spirit and scope of the invention.

What is claimed is:

1. A knee joint prosthesis located between a thighbone and a tibia to couple with a femur implanted component on the thighbone to allow a patient to resume normal movement and exercise, comprising:
a tibial insert which has two concavities for the femur implanted member to move thereon and a moving aperture to hold a bracing member coupled with the femur implanted member, the bracing member having a fastening section; and
a tibial baseplate which has a holding surface to hold the tibial insert and a retaining member which has an anchor section corresponding to the fastening section.

2. The knee joint prosthesis of claim 1, wherein the tibial baseplate further has a retaining flange extended from the holding surface on an edge of one end thereof.

3. The knee joint prosthesis of claim 2, wherein the retaining flange has an inhibiting section, the tibial insert having an anchor wing corresponding to the inhibiting section.

4. The knee joint prosthesis of claim 1, wherein the tibial dock has an implanting strut extended therefrom in an integrated manner to couple with the tibia.

5. The knee joint prosthesis of claim 1, wherein the bracing member and the retaining member are coupled together and swivelable (rotational) in the moving aperture.

6. The knee joint prosthesis of claim 5, wherein the retaining member has a retaining section extended therefrom that is larger than the moving aperture, and the tibial baseplate has an anchor trough for holding the retaining section.

7. The knee joint prosthesis of claim 5, wherein the fastening section of the bracing member has external screw threads and the anchor section of the retaining member has internal screw threads mating the external screw threads.

8. The knee joint prosthesis of claim 1, wherein the fastening section of the bracing member has a moving strut, and the anchor section of the retaining member has a guiding flute for receiving the moving strut and a moving groove communicating with the guiding flute.

9. The knee joint prosthesis of claim 8, wherein the retaining member and the tibial baseplate are integrally formed.

10. The knee joint prosthesis of claim 8, wherein the retaining member and the tibial baseplate have respectively an anchor section and an anchor trough mating each other.

11. The knee joint prosthesis of claim 10, wherein the anchor section has external screw threads, and the anchor trough has internal screw threads mating the external screw threads.
